(19) United States
(12) Patent Application Publication
(10) Pub. No.: US 2015/0351739 A1
(43) Pub. Date: Dec. 10, 2015

(54) FILAMENTARY FIXATION DEVICE AND ASSOCIATED METHODS OF MANUFACTURE AND USE

(71) Applicant: HOWMEDICA OSTEONICS CORP., Mahwah, NJ (US)

(72) Inventor: Anthony P. Napolitano, Chappaqua, NY (US)

(21) Appl. No.: 14/298,295

(22) Filed: Jun. 6, 2014

(51) Int. Cl. A61B 17/04

(52) U.S. Cl. CPC A61B 17/0401

(57) ABSTRACT

A fixation device formed from a single length of filament includes a locking splice forming a first loop disposed at a first end of the fixation device. The device also includes an intermediate segment fixed to and extending away from the locking splice. Further included in the device is a tortuous segment fixed to and extending away from the locking splice. The tortuous segment is slidably engaged to the intermediate segment at a plurality of spaced apart locations along the length of the tortuous segment.

### BACKGROUND OF THE INVENTION

Often sutures and fixation/anchoring devices are utilized in the repair or replacement of soft tissue and/or bony structures. Typically this involves securing a fixation device to bone and tethering the soft tissue and/or bony structures to the fixation device with a suture. In many instances, the suture is tied with a knot, such as a half hitch or the like, to help maintain the tissue and/or bony structures in the desired location during the healing process.

Traditional fixation devices are typically made from metal or hard polymer and require sufficient bulk to be able to withstand the forces applied to the device. Despite their widespread use, such fixation devices are not ideal for certain applications as the bulk of such devices may limit the location of the repair site or render their use impracticable. Additionally, whether such fixation devices are anchored within a bore hole in bone or passed through a bone tunnel and secured against a bone's outer cortex, the bulk of such devices may require the excessive removal of healthy bone in an effort to accommodate their size.

Recent trends have seen the development of “soft” fixation devices, also referred to as “filamentary” fixation devices. While filamentary fixation devices are generally an improvement over the bulkier traditional fixation devices, current filamentary fixation devices can still be constructed to have a smaller size, particularly for certain surgical applications where bone volume is at a minimum. Thus, there is a need for fixation devices with reduced bulk that can be utilized in the repair or replacement of soft tissue and/or bony structures.

### BRIEF SUMMARY OF THE INVENTION

The present invention generally relates to fixation and anchoring devices formed and constructed from a single length of filament. The single length of filament is configured to form a fixation or anchoring device suitable for use in reattaching soft tissue to bone, or other such surgical repairs. Such fixation and anchoring devices may not require the inclusion of any traditional suture or bone anchors in making such repairs. Such fixation and anchoring devices are designed to, for example, be simply positioned in a bore hole in bone and actuated to secure itself within the bore hole.

In a first aspect of the present disclosure, a fixation device formed from a single length of filament includes a locking splice forming a first loop disposed at a first end of the fixation device. The device also includes an intermediate segment fixed to and extending away from the locking splice. Further included in the device is a tortuous segment fixed to and extending away from the locking splice. The tortuous segment is slidably engaged to the intermediate segment at a plurality of spaced apart locations along the length of the tortuous segment.

Additionally, a second end of the fixation device may include a second loop, and the locking splice may be a locked Brummel splice. The second loop may also be formed from a locked Brummel splice. The second loop may also be positioned through and extending out of the first loop to form a third loop.

Continuing with this aspect, the single length of filament may be a braided single length of filament and may include a plurality of pass-throughs spaced apart along the tortuous segment and formed within the braid of the single length of filament. The intermediate segment may extend through the plurality of pass-throughs forming a sliding splice between the tortuous and intermediate segments. The plurality of pass-throughs may be about 2 to 30 pass-throughs. Alternatively, the pass-throughs may be about 2 to 20 pass-throughs, or about 2 to 5 pass-throughs.

Additionally, the intermediate segment may include a first free end, and the tortuous segment may include a second free end. Further, the tortuous segment may be a sinusoidal path of filament having a plurality of bends such that each bend is disposed at an opposite side of the intermediate segment as an adjacent bend.

In another aspect of the present disclosure, a method of forming a fixation device includes the step of forming a locking splice from a portion of a single length of filament such that a tortuous segment and an intermediate segment extend from the locking splice. The locking splice defined a first loop. The method also includes passing the intermediate segment through the tortuous segment at a plurality of positions along the tortuous segment.

Additionally, the locking splice may be a locked Brummel splice. Further the method may include the step of forming a second loop from a portion of the single length of filament adjacent a free end defined by the intermediate segment for ensnaring a working filament. The second loop may also be formed from a locked Brummel splice. The method may also include the step of passing the second loop and at least a portion of the intermediate segment through the first loop, thereby forming a third loop. The passing of the intermediate segment through the tortuous segment may be done at spaced apart intervals along the tortuous segment such that the tortuous segment forms a sinusoidal path of filament having a plurality of bends such that each bend is disposed at an opposite side of the intermediate segment as an adjacent bend.

In a further aspect of the present disclosure, a method of securing a fixation device formed from a single length of filament in a bore hole in bone includes the step of providing the fixation device having a locking splice, an intermediate segment, and a tortuous segment. The locking splice is disposed at a first end of the fixation device. The intermediate segment is fixed to and extends away from the locking splice. The tortuous segment is fixed to and extends away from the locking splice. The tortuous segment is slidably engaged to the intermediate segment at a plurality of spaced apart locations along the length of the tortuous segment. Also included in the method is the step of inserting at least a portion of the tortuous segment into the bore hole with an insertion end of an insertion device. Additionally, the method includes the step of trapping the tortuous segment between the insertion end and locking splice such that the intermediate segment is slidable with respect to the tortuous segment. Another step of the method is tensioning the intermediate segment, while the insertion end temporarily remains in place, such that the locking splice travels toward the insertion end, thereby compressing the tortuous segment within the bore hole.

Additionally, fixation device may also include a second loop disposed at a second end of the fixation device adjacent the intermediate segment. The method may also include the step of ensnaring a working suture with the second loop. Further, the method may include the step of passing the second loop and a portion of the intermediate segment through the first loop to form a third loop. The plurality of spaced apart locations may include pass-throughs formed within the tortuous segment such that slidable engagement with the intermediate segment forms a plurality of slidable splices.

Continuing with this aspect, the bore hole may have a diameter of about 0.75 mm to 2 mm. Also, the bore hole may have a diameter of about 0.75 mm to 1.5 mm. Further, the bore hole may have a diameter of about 0.75 mm to 1.3 mm.

### BRIEF DESCRIPTION OF THE DRAWINGS

These and other features, aspects, and advantages of the present invention will become better understood with regard to the following description, appended claims, and accompanying drawings where:

FIG. 1 illustrates a fixation device in accordance with one embodiment of the present invention.

FIGS. 2-5 illustrate one embodiment of a method of assembly of the fixation device of FIG. 1.

FIGS. 6 and 7 illustrate one embodiment of a method of use of the fixation device of FIG. 1.

FIG. 8 illustrates an alternative embodiment of a method of use of the fixation device of FIG. 1.

FIGS. 9-12 illustrate another embodiment of a method of use of the fixation device of FIG. 1.

FIG. 13 illustrates an anchoring assembly in accordance with one embodiment of the present invention.

FIG. 14 is a schematic top view of a method embodiment for repair of a meniscal tear using the anchoring assembly of FIG. 13.

FIG. 15 is a schematic side view of the method embodiment of FIG. 14.

### DETAILED DESCRIPTION

The fixation devices, assemblies, systems, kits, and associated methods of use of the present invention are intended for use in the repair, reattachment, replacement or otherwise securement of tissue, including both hard tissue (i.e., bone or the like) and soft tissue. Soft tissue may be, for example, meniscus, cartilage, capsule, ligaments, muscle and tendons, replacement grafts of any of these soft tissues, or the like. While many of the exemplary methods disclosed herein are directed towards the use of fixation assemblies and systems involving a filamentary/suture anchor for implantation into a bone hole, other uses, some of which are described herein, are also envisioned. Additionally, the devices, assemblies, systems and methods disclosed herein are contemplated for use in both open surgery and arthroscopic surgery.

As used herein, “proximal” or “proximally” means closer to or towards an operator, e.g., surgeon, while “distal” or “distally” means further from or away from the operator. Also, as used herein, the terms “about,” “generally” and “substantially” are intended to mean that slight deviations from absolute are included within the scope of the term so modified.

As used herein, the term “filament” or “filamentary” is defined as a suture or other thread-like material. Such filaments may be constructed of synthetic material (e.g., PLGA, UHMWPE (ultra high molecular weight polyethylene), polyester, PEEK, Nylon, polypropylene, aramids (for example Kevlar®-based fibers) or the like, or blends thereof), organic material (silk, animal tendon, or the like or blends thereof), or blends of both one or more organic materials and one or more synthetic materials. Alternatively, filaments may include thin metal wires. While any of these materials may be used, it is preferable, and is disclosed herein, that the various filaments or filamentary aspects of the present invention be constructed out of suture, such as UHMWPE, polyester or blends thereof.

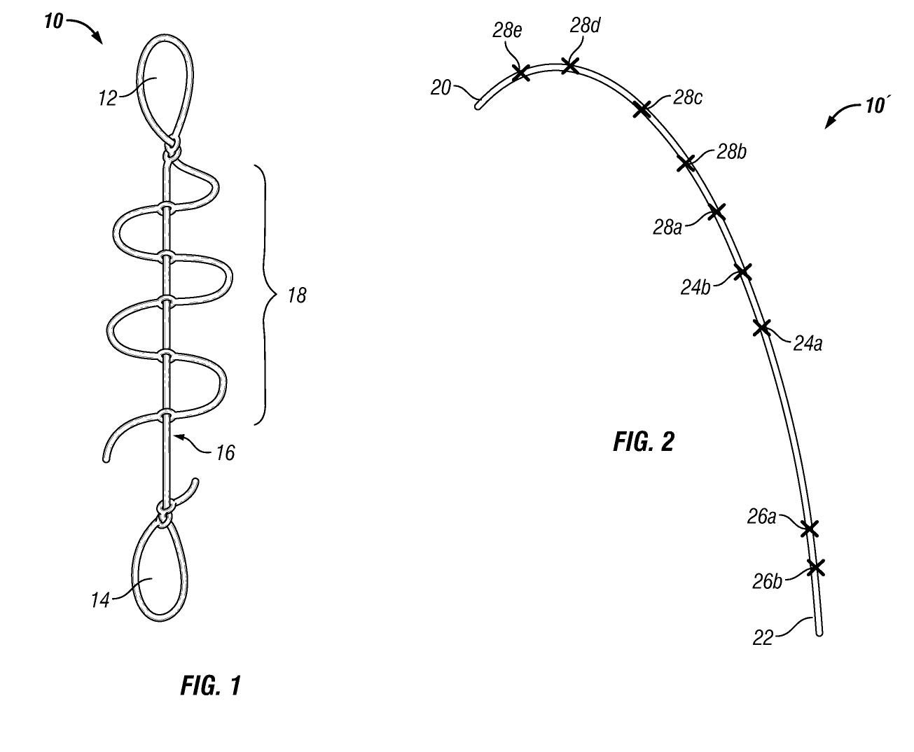

FIG. 1 depicts one embodiment of a fixation/anchoring device 10 that is generally constructed from a single length of filament and includes a first loop 12, a second loop 14, an intermediate segment 16, and a tortuous segment 18. In some embodiments, device 10 may be constructed from multiple lengths of filament or other components as described further below. A locking splice preferably forms the first loop 12 and second loop 14, which are each disposed at a first and second end of fixation device 10, respectively. The intermediate segment 16 and tortuous segment 18 extend away from the first loop 12. The tortuous segment 18 is slidably engaged to the intermediate segment 16 in a generally sinusoidal or serpentine configuration.

FIGS. 2-5 depict a method of assembling anchoring device 10. As previously mentioned, device 10 is generally constructed from a single length of filament 10′ by splicing filament 10′ at multiple locations along its length to transform filament 10′ into fixation device 10. As such, filament 10′ is preferably formed from a braided filamentary material or otherwise includes apertures located along the length of filament 10′ to facilitate spliced formation. As best shown in FIG. 2, filament 10′ includes first and second free ends 20, 22 and a plurality of pass-through locations 24a-b, 26a-b, and 28a-e, also designated by an “X” at each location, which can be gaps within the braiding of the braided filamentary material or apertures formed in braided or non-braided filaments.

Fixation device 10 is assembled by first forming first loop 12, which is preferably a locked loop formed by a locked Brummel splice or the like. The Brummel splice may be formed by any two-tail or single-tail techniques as is known in the art utilizing pass-throughs 24a-b and the first and/or second free ends 20, 22. As illustrated, pass-throughs 24a and 24b are disposed along filament 10′ between pass-throughs 26a-b and pass-throughs 28a-e.

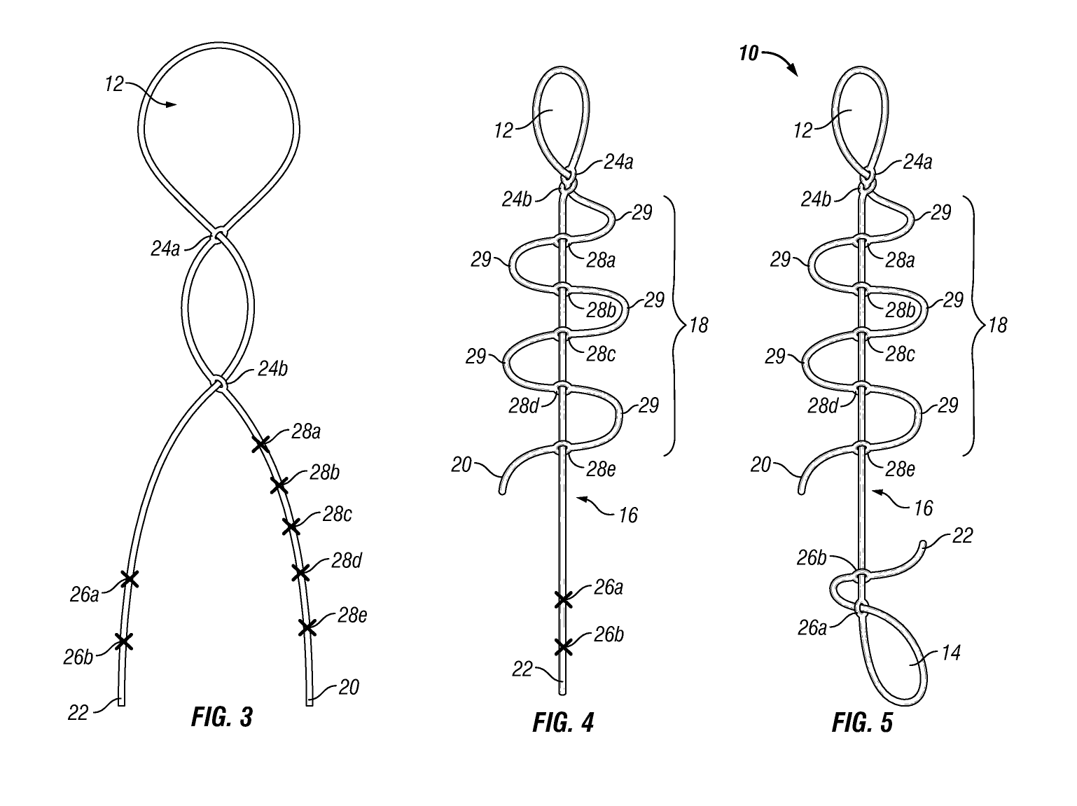

As best shown in FIG. 3, when first loop 12 is constructed, first free end 20 along with pass-throughs 28a-28e extend from first loop 12 adjacent to and in the same general direction as the second free end 22 and pass-throughs 26a-b.

Generally, the locked nature of the Brummel splice or other locking splice provides a fixed abutment region for the tortuous segment 18 to abut as the tortuous segment 18 is slid along the intermediate segment 16 toward the first loop (discussed further below). Additionally, the loop 12 formed by such a splice may be beneficial in that it can be used in conjunction with a lead suture, a hook, or some other leading or retrieving device to facilitate advancement of fixation device 10 through narrow passageways.

However, in some embodiments of fixation device 10 when such a loop is not desired, other techniques may be utilized to achieve the abutment region provided by the Brummel splice or other locking splice without forming a splice and/or first loop, such as loop 12. In one example, an overhand knot, or the like, may be formed along filament 10′ in the general vicinity of pass-throughs 24a-b such that first and second free ends extend from the knot in a similar fashion as previously described. In another embodiment, a small piece of biocompatible polymer may be sonically welded, or otherwise coupled, to filament 10′ in the general vicinity of pass-throughs 24a-b. In yet a further embodiment, a first length of filament having pass-throughs 28a-e and a second length of filament having pass-throughs 26a-b can be coupled at respective ends of each of these filaments by a metallic or polymeric coupling (not shown). The overhand knot, small piece of biocompatible polymer, and polymeric/metallic coupling are just some of the many examples in which an abutment region can be provided for the tortuous segment without forming a loop and/or splice.

As illustrated in FIG. 4, once the first loop 12 is created, the tortuous segment 18 is formed by passing the second free 22 end through pass-throughs 28a-e beginning with pass-through 28a and ending with pass-through 28e to form a generally sinusoidal or serpentine shape. The portion of filament 10′ that extends through pass-throughs 28a-e is intermediate segment 16. Each of the pass-throughs 28a-e is slidable along intermediate section 16 such that the tortuous segment 18 may be transitioned from a first configuration to a second or deployed configuration. The portions of the tortuous segment 18 that connect each pass-through 28a-e are engagement portions 29, which may expand radially outwardly upon the transition from the first to the second configuration.

In the first configuration, the sinusoidal or serpentine shape of the tortuous segment 18 is stretched along the intermediate segment 16 such that the distance between each pass-through 28a-e is greater than in the second configuration. In the second or deployed configuration, the pass-throughs 28a-e are stacked against the Brummel splice formed by pass-throughs 24a-b and bunched together tightly such that each pass-through 28-e touches or nearly touches an adjacent pass-through. Additionally, as a result of the pass-throughs 28a-e being bunched together against the Brummel splice or abutment region, the engagement portions 29 extend further in a radially outward direction when in the second configuration than in the first configuration.

In some embodiments, tortuous segment 18 may have more or less than the five pass-throughs 28a-e depicted. For example, the tortuous segment 18 may have about 2 to 30 pass-throughs. In another example, tortuous segment 18 may include 2 to 20 pass-throughs. In a further example, tortuous segment 18 may include 2 to 5 pass-throughs. In some embodiments, the distance between each pass-through 28a-e may be equal. In other embodiments, the distances may vary. Generally, the more pass-throughs within a constant length tortuous segment, the less radial expansion of the engagement portions 29 when in the second configuration. Conversely, the less pass-throughs within a constant length tortuous segment, the greater the radial expansion.

Once the tortuous segment 18 is formed, the second loop 30 can be formed preferably by another locked Brummel splice, as illustrated in FIG. 5. The Brummel splice may be formed by any single-tail technique known in the art utilizing pass-throughs 26a-b and second free end 22. In some embodiments, the second loop 14 can be formed by other splices, such as an eye splice, or a knot, such as a surgeon's loop knot. In still further embodiments, the second loop 14 can be formed from a second length of filament coupled to the second free end 22 via a knot, mechanical coupling, or the like. The first and second loops 12, 14 formed at each end of fixation device 10 helps contain the tortuous segment 18 to prevent the tortuous segment 18 from becoming disengaged from the intermediate segment 16 during use.

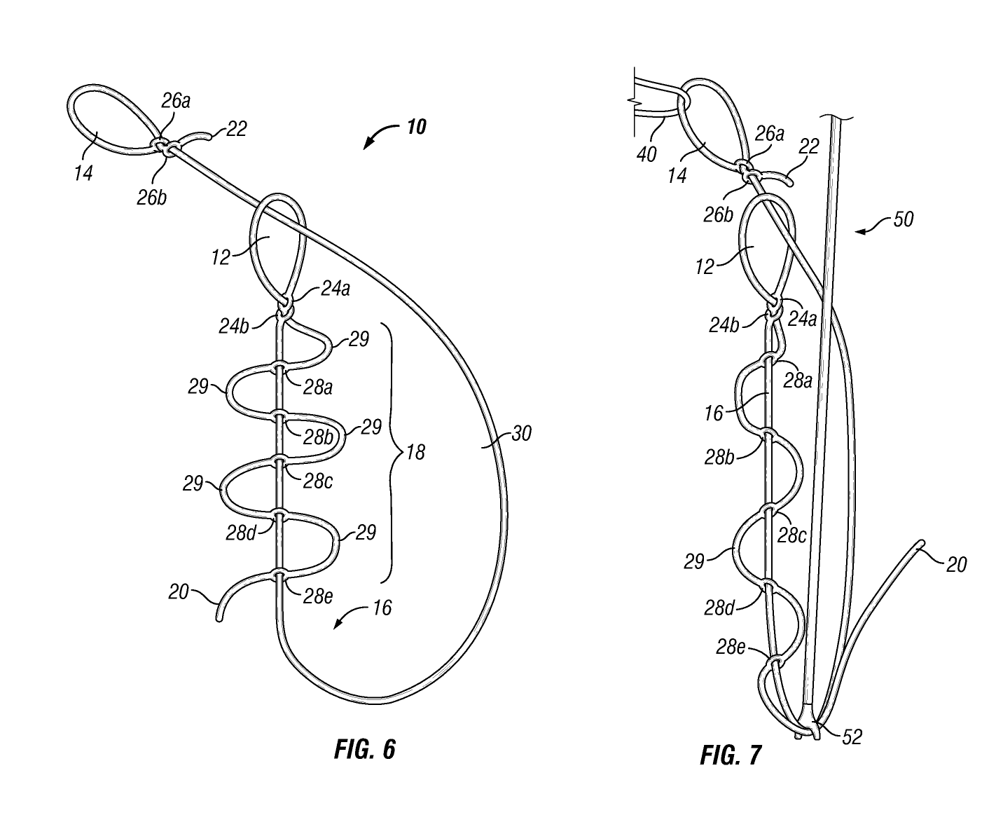

In one embodiment of a method of use, the second loop 14 may be passed through the first loop 12 to form a third loop 30, as best shown in FIG. 6. While this can be performed by the operator during the surgical procedure, passing of the second loop 14 through the first loop 12 can also be performed during the manufacturing process and shipped to the operating room in such a configuration. Thereafter, fixation device 10 may be coupled to an inserter 50, which can also be performed by the operator, or, optionally, during the manufacturing process.

As illustrated in FIG. 7, the inserter 50 may have an insertion end 52 that is horseshoe or goalpost shaped. The insertion end 52 straddles the intermediate segment 16 and the tortuous segment 18 such that the first free end 20 extends from one side of the insertion end 52, and pass-throughs 28a-e extend from another side of the insertion end 52.

During the surgical procedure, a blind bore hole may be drilled into, or otherwise formed, in bone. In one embodiment, the bore hole may have a diameter of about 0.75 mm to 2 mm. In another embodiment, the diameter of the bore hole may be about 0.75 mm to 1.5 mm. In a further embodiment, the diameter of the bore hole may be about 0.75 mm to 1.3 mm. The insertion end 52 along with a portion of fixation device 10 may then be inserted into the blind bore hole.

With the second loop 14 and first free end 20 being controlled by the operator and the insertion end remaining within the bore hole, the operator can tension both the first free end 20 and second loop 14. As tension is applied, the intermediate segment 16 between the first loop 12 and insertion end 52 shortens in length resulting in the first loop 12 moving closer to the bottom of the bore-hole and toward the insertion end 52. The insertion end 52 is shaped such that pass-throughs 28a-e cannot pass to the other side of insertion end 52, which results in the tortuous segment's transition from the first configuration to the second configuration in which pass-throughs 28a-e are bunched together between insertion end 52 and the splice formed by pass-throughs 24a-b.

As the tortuous segment transitions to the second configuration, the engagement portions 29 expand radially outwardly against the bore hole. When fully deployed, the friction created by the expansion of the engagement portions 29 against the wall of the bore hole anchors the filament to the bone. In some embodiments, the bore hole may be an undercut hole in which an undercut portion of the bore hole has a larger diameter than the remainder of the bore-hole. In such an embodiment, the engagement portions 29 may be radially expanded against the wall of the bore hole in the larger diameter undercut portion.

Thereafter, or even during the tensioning process, the inserter 50 may be removed from the bore hole and a working filament 40 in working engagement with the target tissue may be coupled to the second loop 14. In some embodiments, the working filament 40 may be engaged with the second loop 14 prior to insertion of fixation device 10 into the bore hole. Tension from the target tissue is applied to fixation device 10 via the working filament 40, which helps maintain the tortuous segment 18 in a deployed configuration and, therefore, anchored to the bore hole.

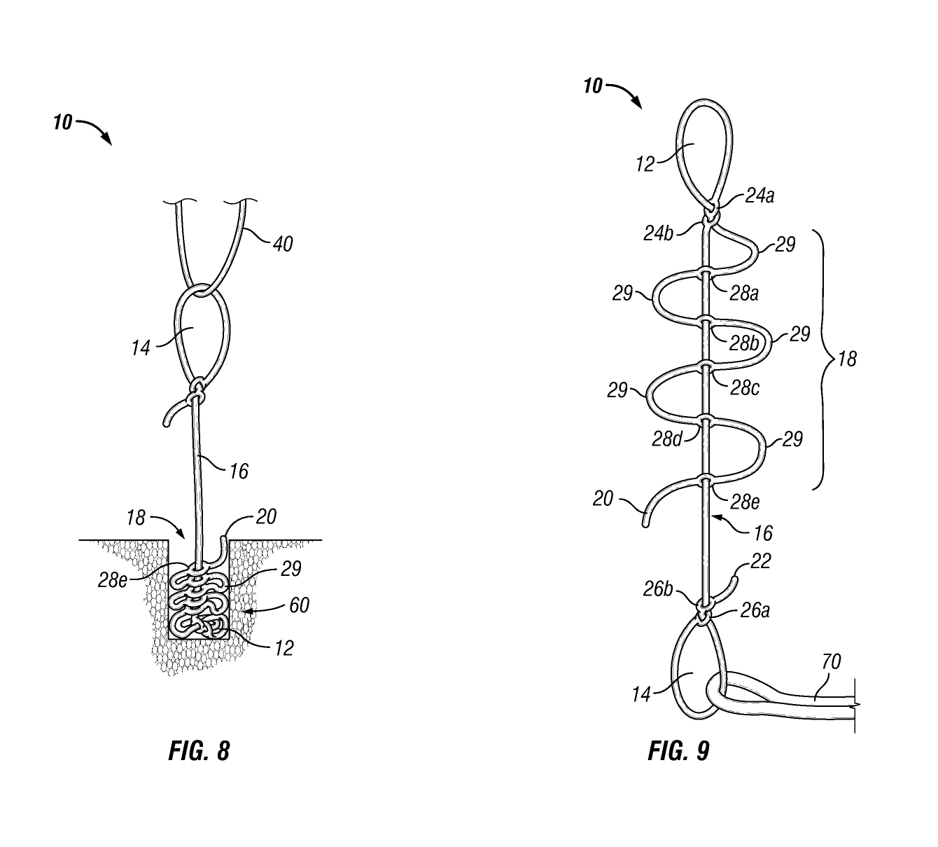

FIG. 8 depicts another embodiment of a method of use in which the second loop 14 is not passed through the first loop as described above. Rather, in this embodiment, an inserter, such as inserter 50 or some other insertion device, may be used via engagement to first loop 12 or some other portion of fixation device 10, to insert the first loop 12 into the bore hole 60. Bore hole 60 is similar to the bore hole described above and may have a diameter of about 0.75 mm to 2 mm. In another embodiment, the diameter of bore hole 60 may be about 0.75 mm to 1.5 mm. In a further embodiment, the diameter of bore hole 60 may be about 0.75 mm to 1.3 mm.

With the first loop in the bore hole 60 and slight tension applied to the second loop 14, a second tool (not shown), such as a knot pusher or the like, may be engaged with the intermediate segment 16 proximal to the tortuous segment 18 and then pushed distally into the bore hole 60. As the second tool is pushed distally, pass-through 28e abuts the second tool and slides toward the first loop 12 adjacent the bottom of the bore hole 60. As this occurs, pass-throughs 28a-e bunch together in the bore hole 60, thereby transitioning the tortuous segment 18 from the first configuration to the second configuration. During this process, the inserter may be removed to allow for the engagement portions 29 to fully expand against the bore hole 60. Thereafter, working filament 40 in working engagement with the target tissue may be coupled to the second loop 14.

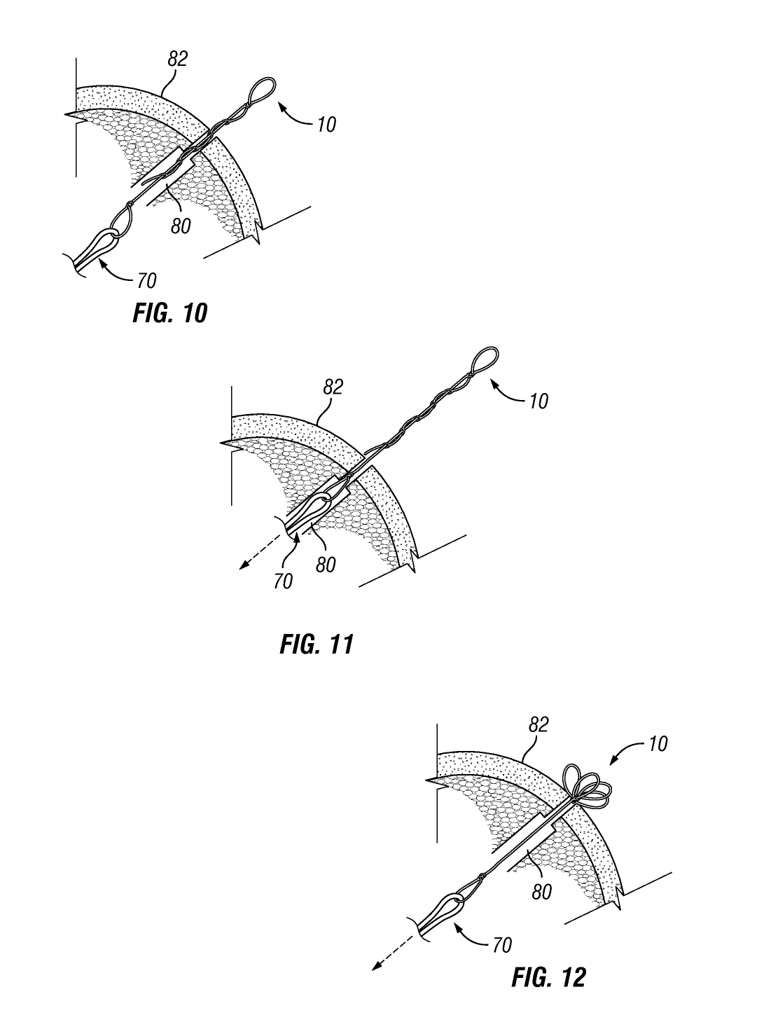

In another embodiment of a method of use as depicted in FIGS. 9-12, fixation device 10 can be used to anchor a tissue graft 70, such as an anterior cruciate ligament (“ACL”) graft, to bone. As depicted, tissue graft 70 can be coupled to the second loop 14 or, in some embodiments, to an adjustable loop device coupled to the second loop. Examples of such an adjustable loop device can be found in U.S. application Ser. No. 13/799,773, filed Mar. 13, 2013 and Provisional Application No. 61/912,307, filed Dec. 5, 2013 both of which are incorporated by reference herein as if fully set forth herein and owned by the same assignee.

Continuing with the example of an ACL repair, the fixation device 10, via a lead suture or the like coupled to the first loop 12, can be navigated through a bone tunnel 80 that extends through to the outer cortex 82 (periosteum or the like) of the femur (or tibia) while the tortuous segment 18 is in the first configuration. In some embodiments, bone tunnel 80 may have a diameter of about 0.75 mm to 2 mm. In another, the diameter of bore hole 60 may be about 0.75 mm to 1.5 mm. In a further embodiment, the diameter of bore hole 60 may be about 0.75 mm to 1.3 mm.

Tortuous segment 18 is passed entirely through the femur, deployed into the second configuration, and positioned on the lateral or outer cortex 82 of the femur. Thus, device 10 may take the place of the traditional “button” anchor, an example of which is found in U.S. application Ser. No. 12/682,324, filed Sep. 7, 2010, incorporated by reference herein as if fully set forth herein and owned by the same assignee. Device 10 can have a smaller shape and may result in less irritation or complications for the patient.

In use, fixation device 10 can act to secure the ligament or replacement graft ligament 70 within the prepared bone tunnel 80 (the preparation of which is described in depth in co-owned U.S. application Ser. No. 13/085,882, filed Apr. 13, 2011 and Ser. No. 12/859,580, filed Aug. 19, 2010). The opposite end of the ligament or graft, which in this example would be positioned within a prepared tibial bone tunnel, can be secured in a similar manner or any otherwise well-known in the art.

Generally, when using fixation device 10 to anchor tissue to a blind bore hole as previously described above, it may be preferable to provide fixation device 10 with relatively numerous pass-throughs, such as pass-throughs 28a-e, and relatively short engagement portions, such as engagement portions 29, when compared to its use with an outer cortex, as also described above. When deployed, shorter engagement portions may have more stiffness than larger engagement portions, which may allow for expansion forces provided by the engagement portions to be more efficiently and effectively applied to the wall of the bore hole. Conversely, relatively large engagement portions may be more susceptible to buckling or bending and may not apply as much force against the wall of the bore hole, which can lead to reduced pull-out strength. Additionally, having numerous pass-throughs takes advantage of the length provided by the bore hole by helping to distribute as many engagement portions along the length of the bore hole as possible to maximize grip.

In contrast, when anchoring fixation device 10 to an outer cortex, it may be preferable to have less pass-throughs with a larger distance between each pass-through than when utilizing fixation device to anchor tissue to a blind bore hole. For instance, as fixation device 10 is passed through a bone tunnel, such as tunnel 80, tortuous segment 18 will generally be in the first configuration allowing for a very narrow bone tunnel, perhaps only slightly larger than double the diameter of filament 10′. When deployed against the outer cortex, the wider expansion of the fewer pass-throughs helps prevent the tortuous segment 18 from reentering the bone tunnel. Moreover, fewer pass-throughs in this application help reduce the height the bunched-up tortuous segment positioned above the bone, which may help to reduce tissue irritation and complication post-surgery.

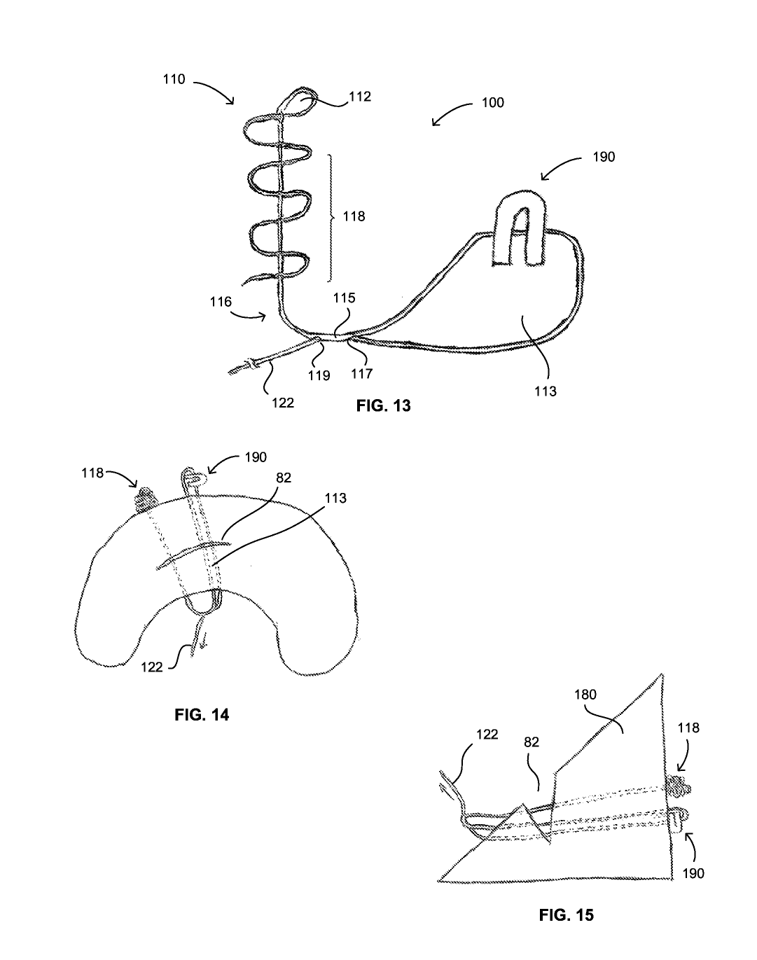

FIG. 13 depicts one embodiment of an anchoring assembly 100. Anchoring assembly 100 generally includes a fixation device 110 and a filamentary sleeve 190. Filamentary sleeve 190 may be constructed from braided filamentary material and may be generally cylindrical in shape with an aperture extending through its length and defining a sidewall. In one example, the filamentary sleeve 190 can be the Iconix® all suture anchor system (Stryker Corporation, Kalamazoo, Mich.). Other configurations are also envisioned, examples of which are disclosed in U.S. application Ser. No. 13/783,804, filed Mar. 4, 2013; Ser. No. 13/303,849, filed Nov. 23, 2011; Ser. No. 13/588,586, filed Aug. 17, 2012; Ser. No. 13/588,592, filed Aug. 17, 2012; and U.S. Pat. Nos. 5,989,252 and 6,511,498, the entireties of which are incorporated by reference herein as if fully set forth herein and all of which are assigned to the same entity as the present invention.

Fixation device 110 is similar to fixation device 10 in that fixation device 110 includes a first loop 112 that may be formed by a locking splice, a tortuous segment 118, an intermediate segment 116, and a free end 122 disposed at an end of intermediate segment 116. However, unlike fixation device 10, free end 122 is threaded through the braiding, or otherwise passed through the sidewall, of filamentary sleeve 190. More specifically, and as shown in FIG. 13, filamentary sleeve 190 is folded over its length into a U-shaped configuration. Free end 122 passes through the sidewall of filamentary sleeve 190 in multiple locations such that filamentary sleeve 190 generally remains in this U-shaped configuration while allowing intermediate segment 116 to slide freely through the sidewall of filamentary sleeve 190. Other configurations of free end 122 and sleeve 190 are also envisioned.

Upon exiting filamentary sleeve 190, free end 122 doubles back to form an adjustable loop 113 and passes into the core of intermediate segment 116 at a first location 117 where free end 122 travels a designated length 115 within intermediate segment 116 and then exits intermediate segment 116 at a second location 119. This length 115 of the intermediate segment through which free end 122 travels may be designated as a “Chinese finger trap” as the braiding of the filamentary material that forms fixation device 10 may have a braided pattern that forms a one-way locking mechanism at least along length 115. In other words, the filament forming fixation device 10 between first and second locations 117, 119 may be braided, or otherwise formed as is known in the art, to allow free end 122 to freely travel through length 115 in one direction, yet is prohibited from travel through length 115 in a second direction.

FIGS. 14 and 15 depict one embodiment of a method of use of anchoring assembly 100 to repair a meniscal tear 82. Anchoring assembly 100 can be used in this regard to form a horizontal or vertical mattress repair as is known in the art. Generally, the first loop 112 is passed through the meniscus and through tear 118 such that tortuous segment 118 extends from meniscus 180. The first loop 112 can be passed through the tissue by any instrumentation known in the art or desired. Tension can be applied at this point to collapse tortuous segment 118 against meniscus 118. Filamentary sleeve 190 is also passed through meniscus 180 and tear 82 at a different location from that of loop 112 using the same instrumentation or different instrumentation as desired. Free end 122 is tensioned, which contracts adjustable loop 113, compresses filamentary sleeve 190 against meniscus 180, and compresses the tissue tear. The “Chinese finger trap” helps prevent assembly 100 from slackening without the need to tie a surgical knot.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

1. A fixation device formed from a single length of filament, the fixation device comprising:
a locking splice forming a first loop disposed at a first end of the fixation device;
an intermediate segment fixed to and extending away from the locking splice; and
a tortuous segment fixed to and extending away from the locking splice, the tortuous segment being slidably engaged to the intermediate segment at a plurality of spaced apart locations along the length of the tortuous segment.

2. The fixation device of claim 1, wherein a second end of the fixation device includes a second loop.

3. The fixation device of claim 2, wherein the locking splice is a locked Brummel splice.

4. The fixation device of claim 3, wherein the second loop is formed from a locked Brummel splice.

5. The fixation device of claim 4, wherein the single length of filament is a braided single length of filament and includes a plurality of pass-throughs spaced apart along the tortuous segment and formed within the braid of the single length of filament, and wherein the intermediate segment extends through the plurality of pass-throughs forming a sliding splice between the tortuous and intermediate segments.

6. The fixation device of claim 5, wherein the plurality of pass-throughs is about 2 to 30 pass-throughs.

7. The fixation device of claim 5, wherein the plurality of pass-throughs is about 2 to 20 pass-throughs.

8. The fixation device of claim 5, wherein the plurality of pass-throughs is about 2 to 5 pass-throughs.

9. The fixation device of claim 5, wherein the intermediate segment includes a first free end and the tortuous segment includes a second free end.

10. The fixation device of claim 4, wherein the second loop is positioned through and extends out of the first loop to form a third loop.

11. The fixation device of claim 1, wherein the tortuous segment is a sinusoidal path of filament having a plurality of bends, wherein each bend is disposed at an opposite side of the intermediate segment as an adjacent bend.

12. A method of forming a fixation device, comprising:
forming a locking splice from a portion of a single length of filament such that a tortuous segment and an intermediate segment extend from the locking splice, the locking splice defining a first loop; and
passing the intermediate segment through the tortuous segment at a plurality of positions along the tortuous segment.

13. The method of claim 12, wherein the locking splice is a locked Brummel splice.

14. The method of claim 13, further comprising:
forming a second loop from a portion of the single length of filament adjacent a free end defined by the intermediate segment for ensnaring a working filament.

15. The method of claim 14, wherein the second loop is formed from a locked Brummel splice.

16. The method of claim 15, further comprising:
passing the second loop and at least a portion of the intermediate segment through the first loop, thereby forming a third loop.

17. The method of claim 16, wherein the passing of the intermediate segment through the tortuous segment is done at spaced apart intervals along the tortuous segment such that the tortuous segment forms a sinusoidal path of filament having a plurality of bends, wherein each bend is disposed at an opposite side of the intermediate segment as an adjacent bend.

18. A method of securing a fixation device formed from a single length of filament in a bore hole in bone, comprising:
providing the fixation device having a locking splice, an intermediate segment, and a tortuous segment, the locking splice being disposed at a first end of the fixation device, the intermediate segment being fixed to and extending away from the locking splice, and the tortuous segment being fixed to and extending away from the locking splice, the tortuous segment being slidably engaged to the intermediate segment at a plurality of spaced apart locations along the length of the tortuous segment;
inserting at least a portion of the tortuous segment into the bore hole with an insertion end of an insertion device;
trapping the tortuous segment between the insertion end and locking splice such that the intermediate segment is slidable with respect to the tortuous segment; and
tensioning the intermediate segment, while the insertion end temporarily remains in place, such that the locking splice travels toward the insertion end, thereby compressing the tortuous segment within the bore hole.

19. The method of claim 18, wherein the fixation device further includes a second loop disposed at a second end of the fixation device adjacent the intermediate segment, and
further comprising ensnaring a working suture with the second loop.

20. The method of claim 19, further comprising the step of passing the second loop and a portion of the intermediate segment through the first loop to form a third loop.

21. The method of claim 18, wherein the plurality of spaced apart locations include pass-throughs formed within the tortuous segment such that slidable engagement with the intermediate segment forms a plurality of slidable splices.

22. The method of claim 18, wherein the bore hole has a diameter of about 0.75 mm to 1.3 mm.
